(19) United States
(12) Patent Application Publication
(10) Pub. No.: US 2004/0110207 A1
(43) Pub. Date: Jun. 10, 2004

(54) Method, computer program product and system for microarray cross-hybridisation detection

(75) Inventors: Johannes Beckers, Neuherberg (DE); Martin Hrabe De Angelis, Neuherberg (DE); Christine Machka, Neuherberg (DE); Matthias Seltmann, Neuherberg (DE); Marion Horsch, Neuherberg (DE); Volkmar Liebscher, Neuherberg (DE)

Correspondence Address: ROPES & GRAY LLP, ONE INTERNATIONAL PLACE, BOSTON, MA, 02110-2624, US

(73) Assignee: GSF - Forschungszentrum fuer Umwelt und Gesundheit GmbH, Neuherberg (DE)

(21) Appl. No.: 10/671,004

(22) Filed: Sep. 25, 2003

Related U.S. Application Data
(60) Provisional application No. 60/414,284, filed on Sep. 27, 2002.

(51) Int. Cl. C12Q 1/68

(52) U.S. Cl. 435/6

(57) ABSTRACT

The present invention provides a method of determining hybridization on a microarry, preferably a DNA-chip.

### RELATED APPLICATIONS

This patent application claims the benefit of U.S. Provisional Application No. 60/414,284 filed on Sep. 27, 2002. The specification of this application is incorporated herein by reference in its entirety.

### BACKGROUND OF THE INVENTION

Arrays of immobilised cDNAs or oligonucleotides are emerging as a universal and versatile tool for the functional analysis of RNA expression profiles (Lipshutz et al., Nat Genet, 21, 20-24 (1999); Lockhart et al., Nat Biotechnol, 14, 1675-1680 (1996); Brown et al., Nat Genet, 21, 33-37 (1999); Science, 270, 467-470 (1995); Beckers et al., Curr Opin Chem Biol, 6, 17-23 (2002)). Gene expression profiling using the DNA-chip technology has proven useful and powerful for the analysis of molecular pathways in the molecular network of the cell. A comprehensive transcriptome analysis in a compendium of yeast mutants has led to the identification of new gene functions and co-regulated syn-expression groups of genes (Hughes et al., Cell, 102, 109-126 (2000)). In Drosophila, the DNA-chip technology has been used to study molecular pathways during metamorphosis (White et al., Science, 286, 2179-2184 (1999)), and in human cancer research expression profiling has provided new insights into pathogenesis and in the classification of tumours (Elek et al., Anticancer Res., 20, 53-58 (2000); Dhanasekaran et al., Nature 412, 822-826; Pomeroy et al., Nature, 415, 436-442 (2002)) and inflammatory diseases (Heller et al., Proc. Natl. Acad. Sci. USA, 94, 2150-2155 (1997)).

Comprehensive genome wide expression profiling has been suggested to be one of the tools in the worldwide effort to annotate the mammalian genome with biological functions (Beckers et al., Curr. Genomics, 3, 121-129 (2002); Nadeau et al., Science, 291, 1251-1255 (2001)). Whereas the current knowledge of gene function is usually limited to single pathways or a small set of target genes, transcription profiling of mouse mutant lines (their organs or derived cell lines) or of mice challenged by infectious disease allows a comprehensive analysis of interactions in global regulatory networks. Several recent reports have successfully used DNA microarray technologies for transcriptome analysis in mice. For example, the transcriptional response to ageing in the mouse brain has significant similarities to that in human neurodegenerative disorders, such as Alzheimer's disease (Lee et al., Nat. Genet., 25 294-297 (2000); Lee et al., Science 285, 1390-1393 (1999)). The differential gene expression in several brain regions and the response to seizure has also been analysed and provided evidence that particular differences in gene expression may account for distinct phenotypes in mouse inbred strains (Sandberg et al., Proc. Natl. Acad. Sci. USA, 97, 11038-11043 (2000)). These and further reports (Porter et al., Proc. Natl. Acad. Sci. USA 98, 12062-12067 (2001); Livesey et al., Curr. Biol., 10, 301-210 (2000); Campbell et al., Am. J. Physiol. Cell Physiol., 280, C763-768 (2001)) have provided the proof-of-principle that despite the complexity of mammalian organs expression profiling is a useful tool to identify pathways associated with particular biological processes in the mouse model system. The reliability of expression profile data obtained in DNA-chip experiments is a major concern for the exact appraisal of differential gene expression (Knight, Nature, 410, 860-861 (2001)). The repetition of experiments (Lee et al., Proc. Natl. Acad. Sci. USA 97:9834-9839 (2000)) and replicates of clones in an array (Lee et al., Proc. Natl. Acad. Sci. USA 97:9834-9839 (2000); Tseng et al., Nucleic Acids Res., 29, 2549-2557 (2001)) are standard procedures often used to support the reliability of expression data. However, such procedures cannot exclude the generation of false data. Artifacts can be due to particular probe sequences and structures that cause cross-hybridisation, or the biased labelling with fluorescent dyes and the label itself. Such false data may therefore be highly reproducible. Another approach is the use of several different sequences corresponding to the same mRNA. The number of such probes for one specific gene may be as high as 40 in commercial microarrays (Li et al., Proc. Natl. Acad. Sci. USA, 98, 31-36 (2001)). This strategy requires a high number of specific oligonucleotides per gene, is expensive, and relies on the presumption that the majority of probes for each gene produce specific hybridisation, which is not valid a priori.

The widely accepted MIAME (Brazma et al., Nat. Genet., 29, 365-371 (2001)) standards (Minimal information required for the analysis of microarray experiments) provide guidelines for the normalisation of expression data and the standardisation of expression results obtained by microarray technologies. However, MIAME standards are applied to sets of expression results at a whole.

### SUMMARY OF THE INVENTION

It is an object of the invention to provide an improved method to verify the quality of an or each individual probe immobilised on an array.

It is a further object to provide a method to verify the quality of each individual probe immobilised on an array in relation to the target RNA used for hybridisation.

It is a further object to provide a method for determining hybridization in at least one probe of a microarray.

It is a further object to provide a method to identify probes of the microarray that produce specific hybridisation signals.

It is a still further object to also provide a computer program product comprising program code means stored on a computer readable medium for performing the computable part of such a method when said program product is run on a computer.

It is a further object to also provide a system which is particularly adapted for carrying out the above-mentioned method.

These objects and further objects are achieved with a method, a corresponding computer program product and a corresponding system as recited in the respective claims.

According to the present invention a method is provided for determining hybridization on a microarray, preferably a DNA-chip, with the following steps: providing a microarray with a plurality of probes; conducting in situ fractionation of hybridised target in at least one probe of the microarray by means of at least one wash with a defined stringency; collecting labelling intensity data, such as fluorescent or radioactive intensity data, at or after the in situ fractionation with a defined stringency; repeating the above steps, wherein in a subsequent cycle the defined stringency is increased; generating a set of data corresponding to at least the stringency and the respective labelling intensity data obtained by each cycle for said cycles; and analyzing the set of data for determining hybridization in at least one probe.

According to a preferred embodiment a fractionation curve is generated which makes it possible to filter out and/or eliminate unreliable data from subsequent analyses.

In a further preferred embodiment a microarray is examined by analyzing a plurality or all probes of said microarray in order to identify probes that produce specific hybridization signals.

The invention moreover provides a corresponding computer program product and a corresponding system.

Generally, the cDNA-chip technology is a highly versatile tool for the comprehensive analysis of gene expression at the transcript level. Although it has been applied successfully in expression profiling projects, there is an ongoing dispute concerning the quality of such expression data. The latter critically depends on the specificity of hybridisation data. SAFE (Specificity Assessment from Fractionation Experiments) is a novel method to discriminate between unspecific cross-hybridisation and specific signals. The inventors applied in situ fractionation of hybridised target on DNA-chips by means of repeated washes with increasing stringencies. Different fractions of hybridised target are washed off at defined stringencies and the collected labelling intensity data at each step comprise the fractionation curve. Based on characteristic features of the fractionation curve, unreliable data can be filtered and eliminated from subsequent analyses. The approach described here provides a novel experimental tool to identify probes that produce specific hybridisation signals in DNA-chip expression profiling approaches. The SAFE procedure significantly improves the efficiency and reliability of RNA expression profiling data from DNA-chip experiments and may be applied to biological material from any source.

It has been shown that melting of dsDNA in solution can be described as a melting curve with sigmoidal shape (Voet et al., Biochemistry, 2^{nd} ed. J. Wiley & Sons INc., NY, pp 862-863 (1995)). In such experiments it was proven that for specified solutions the melting temperature depends on the DNA sequence and is maximal for full-length perfect matches. Thus, it is possible to assess the extent of specific hybridisation and cross-hybridisation by measuring melting curves over increasing hybridisation or washing stringencies. In some early applications of microarray technologies it was pointed out, that such “melting curves could provide an additional dimension to the system and allow differentiation of closely related sequences” (Stimpson et al., Proc. Natl. Acad. Sci. USA 92, 6379-6383 (1995)). Subsequently, similar methods were used for mutation diagnostics in the beta-globin gene (Drobyshev et al., Gene, 188, 45-52 (1997)), for the determination of on-chip DNA duplex thermodynamics (Kunitsyn et al., J. Biomol. Struct. Dyn., 14, 239-244 (1996); Fotin et al., Nucleic Acids Res., 26, 1515-1521 (1998)), and for the highly parallel study of DNA interactions with low molecular weight ligands (Drobyshev et al, Nucleic Acids Res. 27, 4100-4105 (1999)) and proteins (Krylov et al., Nucleic Acids Res. 29, 2654-2660 (2001)). However, this principle has until now not been applied to the most popular application of microarrays, the expression profiling technology, using DNA-chips.

Here we use this method to examine probe specificity on a custom made DNA glass chip in combination with different pools of target sequences isolated from a set of different mouse tissues. We present a novel approach providing precise information about the specificity of hybridisation for each probe (also called feature) of an array. The SAFE protocol (Specificity Assessment from Fractionation Experiments) is based on the washing of microarrays with increasing stringencies and the recording of the hybridisation signal intensity for each array element at each step. In case there are different fractions of target hybridised to the same probe, these will be washed off from the array at various stringencies due to different extends of double strand formation. The set of such data for each array element comprises the fractionation curve, which provides novel information that can be used to evaluate hybridisation data reliability.

### Materials and Methods

Tissue Collection

Breeding of wildtype C3HeB/FeJ mice was done under specified pathogen free (spf) conditions. Organs were collected at the age of 105 days (+/−5 days). To minimise the influence of circadian rhythm on gene expression, mice were killed between 9 am and noon by carbon dioxide asphyxiation. Organs (kidney, testis, brain, seminal vesicles) were dissected, weighed, snap frozen and stored in liquid nitrogen until isolation of total RNA.

Embryos were dissected at E10.5 in ice-cold phosphate buffered saline (PBS). Chorion tissue, yolk sack and amnion were removed. Dissected embryos were stored at −80° C. until isolation of total RNA.

Isolation of Total RNA

All reagents were purchased from Sigma-Aldrich, unless otherwise specified. Total RNA was isolated just before processing for expression profiling. For preparation of total RNA individual organs were thawed in buffer containing chaotropic salt (RLT buffer, Qiagen) and homogenised with a Polytron homogeniser. Total RNA from individual samples was obtained according to manufacturer's protocols using either RNeasy Mini or Midi kits (Qiagen). The concentration of total RNA was measured by OD_{260/280} reading. Aliquots were run on a formaldehyde agarose gel to check for RNA integrity. The RNA was stored at −80° C. in RNase free water until fluorescent labelling.

Reverse Transcription and Fluorescent Labelling

For labelling 40 μg total RNA from individual tissues was used for reverse transcription and indirect fluorescent labelling. This was done using either a glass fluorescence indirect labelling kit (Clontech) with minor modifications of the manufacturer's protocol or the aminoallyl labelling of RNA for microarrays following the TIGR protocol (http://atarrays.tigr.org/PDF Folder/Aminoallyl.pdf). Modifications to the Clontech protocol included an extension of the reverse transcription reaction to at least 1 h and a final ethanol precipitation of labelled DNA at −80° C. for 2 h.

Preparation of Probe/Clone Set

The 20,000 (20K) cDNA mouse arrayTAG set (Lion Bioscience) was used to produce bacterial lysates by inoculating bacterial cultures with a 96-needle replicator. The bacteria were grown in 1 ml LB medium in the presence of 100 μg/ml ampicillin at 37° C. in 96 deep-well blocks sealed with airpore sheets (Qiagen) for 24 h in a shaker. For lysates 25 μl of the bacterial cultures was mixed with 75 μl water and incubated at 95° C. for 10 min. After centrifugation at 4000 rpm for 5 min, 5 μl of the lysate supernatant was used for PCR. 95 μl PCR master-mix were added and probes were amplified.

PCR and DNA-Microarrays

Probes were amplified using standard PCR protocols in a Tetrad thermocycler (MJ Research) with 37 cycles (30 sec at 95° C., 30 sec at 52° C. and 1 min at 72° C.) with 5′ amino-tagged primers (forward 5′-NH₂ GTT TTC CCA GTC ACG ACG TTG-3′, and reverse 5′-NH₂ TGA GCG GAT AAC AAT TTC ACA CAG-3′, MWG-Biotech) from the non-redundant and sequence-verified Lion mouse arrayTAG™ 20K clone set. PCR products were amplified to a minimum concentration of 75-100 μg/μl in 99.9% of the clones. All 20,000 probes were quality checked by agarose gel electrophoresis. In the entire set only 7 clones did not amplify and 10 clones showed multiple bands, confirming the high quality of this particular set of mouse clones.

Clones were dissolved in 3-fold SSC and spotted on aldehyde-coated slides (CEL Associates) using the Microgrid TAS II spotter (Biorobotics) with 48 Stealth™ SMP3 pins (Telechem). Spotted slides were rehydrated overnight in a humid chamber containing 50% aqueous solution of glycerol. Rehydrated slides were dried again, immersed in blocking solution (0.1 M sodium borohydride in 0.75 fold PBS with 25% ethanol) for 5 minutes, boiled in water for 2 minutes, briefly immersed in 100% ethanol and air-dried. Slides were stored in slide boxes at ambient temperature until hybridisation.

Hybridisation, Washing, and Image Analysis

DNA microarrays and glass cover slips (Erie Scientific) were pre-hybridised for 45 minutes at 42° C. in pre-hybridisation buffer (6-fold SSC, 1% BSA, 0.5% SDS). After this pre-hybridisation the slides were rinsed in water, ethanol, and air-dried. 45 μl of hybridisation solution (40 μg of each type labelled cDNA in 6×SSC, 0.5% SDS 5 fold Denhardt's solution and 50% formamide) were placed on the slide and covered with cover slip. This assembly was placed into a hybridisation chamber (Gene Machines, USA) and immersed in a thermostatic bath at 42° C. for 22-27 hours. After hybridisation slides with cover slips were immersed in 40 ml of 1×SSC pre-warmed at hybridisation temperature and vigorously shaken to detach cover slips. Slides were rinsed in 1×SSC and ½×SSC at room temperature and placed in a petri dish with ¼×SSC. Slides were trimmed to the length of 46 mm.

A Gene Frame® 19×60 mm microarray sealing spacer (AB Gene) was attached to another cover slip (Erie Scientific), immersed in ¼×SSC in a petri dish with the hybridised slide and pasted to it such that the slots at the top and bottom of the slide were not sealed (since this is 46 mm in length, 14 mm shorter than the cover slide) (FIG. 1).

This assembly was placed into a microarray scanner (GenePix 4000A, Axon) and the image was scanned at both wavelengths (532 nm and 635 nm). 700 μl of ¼×SSC were pipetted to one of the unsealed edges of the slide while the excess of solution was removed from the opposite unsealed side with filter paper. Then the slide was washed in the opposite direction with another 700 μl of the same solution. Further washes were done with increasing concentrations of formamide (in 3.5% steps) in the same ¼×SSC buffer. The range of formamide concentrations was from 0 to 94.5%. After each washing the slide was incubated for 5 minutes and scanned again.

The scanned images of hybridized Microarrays were processed with the GenePix Pro 3 image analysis software. The mean pixel intensities for each single feature obtained after each washing step were plotted versus the stringency as fractionation curves.

Quantitative, Real-Time PCR

Differential expression of selected candidate genes was verified by quantitative PCR (qPCR). qPCR was done using a Light Cycler (Roche) and the FastStart SYBR Green kit (Roche). In brief, 1 μg of total RNA was mixed with 1 μl 0.1 mM random nonamers in a volume of 11 μl, heat denatured for 5 min at 70° C. and chilled in ice water. 4 μl 5×first strand buffer (LifeTechnologies), 2 μl DTT (LifeTechnologies), 1 μl RNase inhibitor (40 U/μl, Roche), 1 μl 4dNTP mix (10 mM, Amersham Biosciene) and 1 μl SuperScriptII (LifeTech) were added and incubated at 42° C. for at least 1 h. After the reaction, the enzyme was heat inactivated for 15 min at 70° C. and the obtained cDNA diluted 1:5 with water. qPCR reactions were done by mixing 2.4 μl 25 mM MgCl₂, 2 μl primer mix (5 mM each) and 2 μl SYBR Green/enzyme mix to a total volume of 18 μl with water, transferring the solution to a microcapillary (Roche) and adding 2 μl of the cDNA template. Primers were designed to be 20 bp in length with a GC content of 55% to amplify a PCR product of a maximum of 200 bp spanning an intron whenever possible. Primers from the mouse HPRT and mouse PBGD “housekeeping” genes were used as internal controls. Cycling conditions were 10 min at 95° C. for activation of the hot start Taq polymerase followed by 45 cycles of 20 sec at 95° C., 20 sec at 55° C. and 10 sec at 72° C. each.

Sequencing and Calculation of Melting Temperature

22 clones/probes were selected for sequencing to enable calculation of melting temperatures. Clones were PCR-amplified in the same manner as for microarray spotting and sequenced (MWG-Biotech) in both directions using the same primers. For the calculation of melting temperatures vector sequences were excluded from the clone sequence and differential melting curves were calculated according to Poland's algorithm (Poland, Biopolymers, 13, 1859-1871 (1974)) in the implementation described by Steger (Steger, Nucleic Acids Res., 22, 2760-2768 (1994)) using the on-line program available at http://www.biophys.uni-duesseldorf.de/local/POLAND/poland.html with thermodynamic parameters (Blake et al., Nucleic Acids Res., 26, 3323-3332 (1998)) for 0.75 mM NaCl and 1 μM strand concentration. The temperature of the final peak on the differential melting curve was taken as the melting temperature of the clone.

### Results

Comprehensive Assessment of Fractionation Curves

As a first step towards the identification of specific and non-specific probes on our 20K DNA-chip, we measured post-hybridisation signal intensities of every feature in situ after gradual increase of washing stringencies (FIG. 1). The result is a unique curve of hybridisation signal intensities depending on washing stringency conditions for each combination of an individual probe and a pool of target sequences isolated from a particular tissue. Signal intensities were recorded after washes with formamide in the range of 0% to 94.5% in steps of 3.5%. We used formamide to manipulate washing stringencies instead of heating, since in our experimental set up this allowed a precise control of washing stringencies. The resulting set of such fractionation curves was examined by means of hierarchical clustering using the Cluster software available from http://rana.lbl.gov/EisenSoftware.htm. Prior to clustering, artifacts that were due, for example, to contamination with dust particles during washing were filtered.

In the experiment shown in FIG. 2 a total of 8980 spotted probes produced a hybridisation signal that was sufficiently strong to be detected by the image analysis software. Microarray features that were not detected by the image processing software were not clustered. A selection of data for Cy5-labelled testis cDNA is presented in FIG. 2. 48% of probes showed a sharp transition from the hybridised to dehybridised state within less than 15% formamide. The stringency at which the transition occurred ranged from 40% to 70% formamide. Typical examples with transition stringencies at 62% and 55% formamide are shown in FIGS. 2A, C and FIGS. 2B, D, respectively. For 29% of probes the accuracy of fractionation curves was insufficient to draw a conclusion about the character of transitions due to relatively weak signals and high noise (not shown). The remaining 23% of clones revealed different shapes of fractionating curves, such as two-step fractionation curves (FIG. 2F), broad transition regions (FIG. 2E) and a variety of intermediate shapes (not shown). To confirm that bleaching after repeated scans of the hybridized arrays did not significantly contribute to the fractionation curves, fluorescently labelled oligonucleotides complementary to primer sequences were hybridised to the array. After 30 scans the spot intensity was on average 72% of the initial signal intensity (not shown). Taking into account that the transition from hybridized to dissociated target molecules usually occurred over 6 scanning/washing intervals, bleaching did not significantly contribute to the shape of fractionation curves. Based on established hybridisation behaviour in solution, we hypothesized that fractionation curves with two-step (FIG. 2F) or broad transition (FIG. 2E) may be indicative of two or more target molecules that hybridise to these probes. In contrast, we suggest that sharp transitions (FIGS. 2C and D) are a prerequisite for the specific hybridisation with one particular target cDNA or with cDNAs that are highly homologous over the length of the probe.

Transition Stringencies as Characteristic Feature of Fractionation Curves

A major characteristic parameter of the fractionation curve is the transition stringency, which is defined as the midpoint of the transition region (e.g., 62% formamide for the fractionation curves in FIG. 2C, 55% formamide in FIG. 2D). Transition stringencies were highly reproducible for each probe in independent experiments, on separate DNA-chips, with different labels but from the same tissue of different individual mice. As an example, the correlation of transition stringencies (expressed as % formamide) for kidney cDNA labelled with different fluorescent dyes and hybridised to separate slides in independent experiments is shown in FIG. 3. These data have a correlation coefficient of 0.95 and a standard deviation from the best fit of 1.6% formamide. This shows that the transition stringency is a characteristic and reproducible parameter of a probe in combination with defined pools of target molecules.

Transition Stringencies as Major Criteria for Probe Specificity

We use the comparison of transition stringencies of individual probes in hybridisation experiments of different tissues as measure of probe specificity. Since a full-length perfect match between probe and target is the most stable DNA duplex that can be formed, it has the maximal transition stringency. In the case of mismatched or partial hybridisation, which occurs in cross-hybridisation, the transition will take place at a lower stringency. Here we use the reduced transition stringency as an indicator of non-specific hybridisation: if for a particular clone the transition stringency is lower for the cDNA from one tissue as compared to a reference tissue, and if this is confirmed in a colour flip experiment (switching the fluorescent labels), then we conclude that this clone produces non-specific hybridisation with the cDNA pool from the experimental tissue.

To compare transition stringencies and to address the question of probe specificity we hybridised a set of cDNAs isolated from different mouse tissues that is routinely used in the analysis of expression profiles from mutant mouse lines. As an example, the analysis of transition stringencies from hybridisations with cDNAs from whole embryos (E10.5) and adult testis is shown (FIG. 4). To normalize fractionation curves of individual probes we first calculated the median signal intensities for all probes on the microarray over increasing stringency (FIGS. 4A and B, showing the corresponding colour flip experiments). The data shown represent the normalized median over all spots detected by the image processing software. The data were normalized by subtracting the residual signal intensities from all measuring points such that the median of the last 7 measuring points (at high stringency) was set to 0. In addition, signal intensities from all measuring points were multiplied by a scaling factor such that the median signal intensities of the first 7 measuring points (at low stringency) was 1. Thus, FIG. 4A shows the normalized, median fractionation curve over all gene expression detected in embryo (red) and testis (green). FIG. 4B shows the corresponding result in the colour flip experiment. Whereas the shapes of the median fractionation curves are similar and reproducible in both tissues, we find that transition stringencies are slightly increased by approximately 2% formamide for the green fluorescent dye. This difference is comparable to the spread of transition stringencies in FIG. 3 and is not significant for the subsequent analysis of transition stringencies of individual probes.

An example for the analysis of transition stringencies for individual probes is illustrated in FIGS. 4C and D for the probe corresponding to the mouse HSP40 gene. The fractionation curves for this gene were normalized by subtracting the same residual signal intensity at high stringency and multiplying by the same scaling factor as in FIGS. 4A and 4B, respectively. The data show that the HSP40 transition stringency for cDNA from embryo tissue is significantly lower (by ˜20% formamide) as compared to the transition stringency for testis cDNA (FIG. 4C). This finding was confirmed in the corresponding colour flip experiment (FIG. 4D). The initial, normalized signal intensity for embryo cDNA was 60-65% of the intensity for testis cDNA in both experiments. Thus, based on the gene expression data in a normal expression profiling experiment (corresponding to the measurement at 0% formamide) it would have been estimated that HSP40 in embryo is expressed at 60-65% of the level in testis. However, the reduced transition stringency of HSP40 in embryo indicates that this signal results from extensive cross-hybridisation: at a stringency of 63% formamide the signal intensity resulting from embryo cDNA was at background level, while the decrease of the testis signal was less than half the initial signal intensity. This corresponds approximately to a 10-fold difference in the ratio of signal intensities in the transition region of the specific hybridisation in testis (63% formamide, FIGS. 4E and F).

Verification of Cross-Hybridisation by qPCR

We used quantitative real-time PCR to verify that expression of HSP40 in the embryo is indeed less than 60-65% of the expression in testis (FIG. 5). These data suggest that during the exponential phase of the PCR amplification, the background-corrected signal intensity for HSP40 in testis (FIG. 5, thick blue line) is approximately 13 times higher than for embryo tissue (FIG. 5, thick brown line). If the data is normalized with respect to a housekeeping gene, such as HPRT (FIG. 5, thin brown and blue lines), the testis/embryo ratio for the HPS40 gene is ˜65 fold. Regardless of the normalisation procedure, the real-time quantitative PCR supports that expression of HSP40 in testis versus embryo is significantly higher than suggested by a standard DNA-chip experiment.

Towards a Comprehensive Approach to Estimate Cross-Hybridisation

To begin to comprehensively assess the specificity of probes used on our 20K mouse DNA-chip we compared transition stringencies from total RNA isolated from a subset of organs that are routinely used in the analysis of expression profiles of mouse mutant models. The organs analysed in this study comprise adult kidney, testis, brain, seminal vesicles, and whole embryos (E10.5). To analyse fractionation curves we performed pair-wise hybridisations of these organs (FIG. 6), including the corresponding colour flip experiments. Transition stringencies were compared in both experiments, using the ratios of signal intensities over increasing stringency (as in FIGS. 4E and F).

This analysis is reasonable only if the signal intensity of both fractionation curves is high and a sigmoidal shape is clearly detectable. In particular, signal intensities close to background levels would lead to division by zero or produce high noise. Therefore, for the comparison of transition stringencies in different tissues, we selected only those probes having a mean signal intensity above a specific threshold for both wavelengths (i.e., Cy5 and Cy3). This threshold was 150 arbitrary fluorescence units for both hybridisations in experiment #1, 200 units for experiments #2 and #4, and 150 units in one hybridisation of experiment #3 and 400 units in the corresponding colour flip hybridisation of experiment #3. For example, in experiment #1 (embryo/testis) we identified 4452 genes that were expressed above this threshold in both tissues and in both corresponding colour flip experiments. 1456 such genes were identified between embryo and kidney (experiment #2), 748 between testis and seminal vesicles (experiment #3), and 3171 between brain and kidney (experiment #4) (FIG. 6, last column).

Exclusion of Non-Specific Hybridisation

To identify probes among them that result from non-specific hybridisation we compared transition stringencies between tissues. As a measure for the difference in transition stringencies we evaluated the ratio curves (as in FIGS. 4E and F). Each ratio curve with a peak of at least 1.4 relative to the median of the curve was verified individually. For example, in experiment #1 64 probes with a transition stringency that was significantly lower in total RNA isolated from embryo as compared to total RNA from adult testis were identified (FIG. 6, left column). In turn, for testis RNA 10 probes were identified with reduced transition stringencies as compared to embryo RNA (FIG. 6, left column). The probes listed in the left column of FIG. 6 have been annotated as resulting in non-specific hybridisation in the corresponding tissue. The limited data presented here, suggests that at least 0.2% (10/4452, testis, experiment #1) to 1.7% (13/748, seminal vesicles, experiment #3) of the probes evaluated by the criteria described above produce signals that result from unspecific hybridisation. However, the portion of such unspecific probes is most likely significantly higher. It would be required to compare fractionation curves of more tissues, since transition stringencies could be decreased for both tissues used in one hybridisation experiment. As an example, in experiment #2 the transition stringency of the HSP40 gene was at 49% formamide for both embryo and kidney, while in experiment #1 it was 46% formamide for embryo and 65% formamide for testis (FIG. 4C and D). Therefore, only experiment #1 was suitable to identify the HSP40 probe as unspecific for the assessment of expression in embryo RNA.

In addition, a significant number of probes had decreased transition stringencies in one fractionation curve, while for the colour flip hybridisation the signal was too weak to determine the transition stringency (FIG. 6, middle column). This finding could be due, for example, to minor variations in hybridisation conditions. It is likely that such probes may also produce signals that result from unspecific hybridisation.

Comparison of Melting Temperatures and Transition Stringencies

It may be expected that probes with transition stringencies below a particular threshold should be considered as resulting in cross-hybridisation. To verify this, 22 probes present on our array were fully sequenced and their theoretical melting temperatures were calculated. To evaluate their correlation, these melting temperatures were plotted versus their transition stringencies measured in experiment #1 (FIG. 7). Nine of the 22 selected probes had significantly different transition stringencies in testis and embryo RNA (FIG. 7, white squares, lower transition stringencies). The correlation plot from probes with equal/maximal transition stringencies in both tissues (black squares) describes a different region in the graphic (separated by dotted line) than those with reduced transition stringencies (with one exception, which is most likely due to the fact that the measured transition stringency for this probe is not maximal, similar to the low transition stringency of HSP40 in both tissues of experiment #2). However, there is a correspondence between calculated melting temperatures and the maximal measured transition stringencies (black squares, region above dotted line). This characteristic may be useful for the evaluation of the specificity of hybridisation based on the measurement of transition stringencies from single tissue RNAs and the sequence of the probe, without the measurement of transition stringencies in relation to other reference RNAs.

### Discussion

Although the DNA-chip technology has been applied successfully for expression profiling projects (see introduction), there is an ongoing dispute concerning the quality of expression data that can be obtained from such experiments. It is known from practical experience with established hybridisation technologies, such as Northern-, Southern-blot, and in situ hybridisation methods, that the quality of the data obtained in these approaches critically depends on the selection of probes that specifically hybridize to the target mRNA. Whereas in single gene approaches it is possible to assess probe specificity empirically, this has until now not been feasible for genome wide sets of probes. Theoretical considerations such as avoiding repetitive sequences and conserved functional domains of paralogous genes have been suggested as criteria for the selection of specific probes. The applicability of this strategy depends on the completeness of sequence information. Another approach, used also for the clone set in the study described here, utilises probes that are preferentially derived from 3′ untranslated regions. Using the SAFE protocol, we provide here, for the first time, a method to assess probe specificity at large-scale based on experimental hybridisation data.

Technically expression profiling using DNA-chips is similar to the procedures of the classical dot-blot: Gene specific oligonucleotides or double-stranded cDNAs are immobilized as probes in defined positions on a solid support and hybridized to complex mixtures of expressed nucleic acids. Using the current standards of microarray spotters, up to 50 thousands spots may be fitted on a standard chip of the size of a common histological slide. An important advantage of using glass as transparent, solid support is that it allows the simultaneous, competitive hybridization of test and reference samples labelled with different fluorescent dyes. Relative expression levels are analyzed directly by comparing each fluorescent signal on every feature. An additional advantage of the DNA-chip technology, as compared to other expression profiling methods such as SAGE (serial analysis of gene expression), is that the production, hybridization, and scanning of such DNA-chips can be automated to a great extend allowing for high-throughput approaches.

The hybridisation specificity of probes depends on the population of target molecules that compete for hybridisation with the nucleotide sequence of the probe and on the stringent condition that is used in the experiment. A probe that produces a specific signal in a hybridisation experiment with total RNA from one tissue may show extensive cross-hybridisation with total RNA from another tissue that expresses other populations of genes. We demonstrate that reduced transition stringencies determined in fractionation curves of simultaneous hybridisation experiments with RNAs from different tissues are indicative of unspecific hybridisation signals. This tissue-related information about the probe specificity is an efficient tool to validate data on differentially expressed candidate genes based on attributed weights or confidence in the probe. Using the experimental set-up described here, the measurement of fractionation curves on DNA glass slides takes approximately 5 hours for a single hybridisation experiment. To fully implement the validation of probe specificities based on fractionation curve data it would be required to measure transition stringencies in a combinatorial way using a considerable set of different RNA pools. For example, we apply the DNA-chip technology to systematically analyse expression profiles of a selection of 17 mouse organs in a compendium of several hundred established mouse mutant lines (Hrabe de Angelis et al., Nat. Genet. 25, 444-447 (2000)). The comprehensive assessment of transition stringencies in this set of RNA pools would require the experimental measurement of 136 pairs of tissues in at least two experiments (i.e., the corresponding colour flip hybridisations). The further automation of measuring fractionation curves and developing algorithms to analyse transition stringencies would make it feasible to estimate probe specificities on DNA-chips at large scale.

Such comprehensive analyses of fractionation curves will result in the identification of reliable probes for expression profiling studies using the DNA-chip technology. This approach could ultimately be used to identify reliable probes for each gene that result in high quality expression data in a wide range of RNA pools from different resources. The data presented here (in particular, in FIG. 6) provides a first step towards this goal. To complete this data set we are currently developing reliable software tools for the calculation of transition stringencies from fractionation data.

In addition, we provide evidence that transition stringencies that result from specific hybridisation signals (maximal transition stringencies) correlate well with the calculated melting temperature of the corresponding probe sequence (FIG. 7). Thus, the comparison of the experimentally measured transition stringency with the calculated melting temperature of a full-length hybridisation with the probe provides an additional means to estimate potential probe specificity. In contrast, to the full experimental approach described above, this method does not rely on measuring differences between diverse RNA pools. Instead, the transition stringency measured in a single experiment may be compared to the theoretical melting temperature to assess probe specificity.

The correlation of melting temperatures and formamide stringencies at which the transition from hybridized to non-hybridized target molecules occurs is a phenomenological observation that we made in the course of this study. Although, such a correlation may have been expected (Blake et al., Nucleic Acids Res., 24, 2095-2103 (1996)), an adequate physical model does not underline it. It implies that an increase in temperature during washing steps has the same effect as an increase in stringency by elevating formamide concentrations. It also does not take into account that melting temperatures are calculated for dsDNA in solution, whereas fractionation curves are measured with probes that are immobilized on a solid surface. Although the influence of these factors may not be significant for measuring transition stringencies in the majority of cases a proper physical model should be elaborated. Alternatively, the accuracy of fractionation curve measurements could be further improved by detecting signal intensities in situ during washing conditions with increasing temperature instead of formamide concentrations. However, this is not possible with currently available microarray scanners and would require considerable changes in the technological set up.

The SAFE protocol described here, provides a novel tool for the assessment of probe specificity used in genome wide DNA-chip expression profiling experiments. These procedures will allow the selection of specific probes that will lead to high quality expression profiling data resulting from DNA-chip experiments.

### DESCRIPTION OF THE FIGURES

FIG. 1: Scheme of experimental set-up (see Materials and Methods for description).

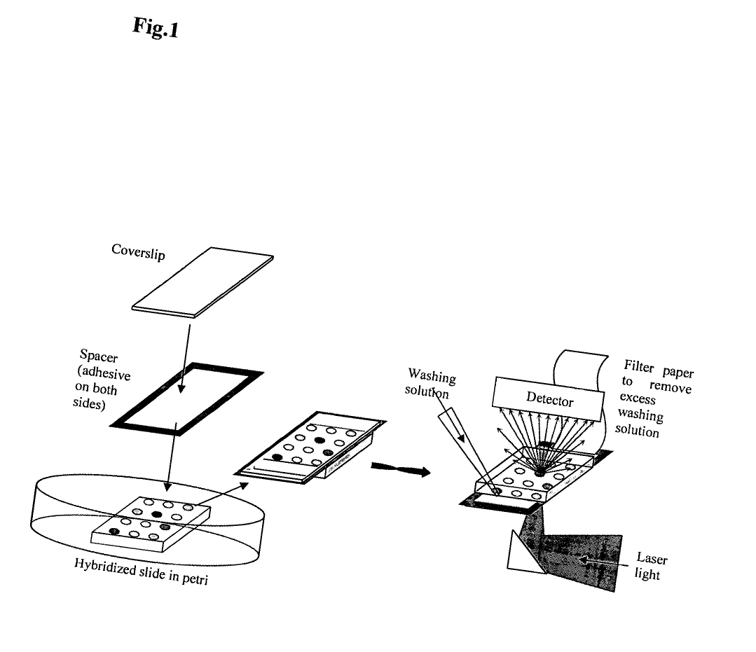

FIG. 2: Comprehensive assessment of shapes of fractionation curves from normalized data. Fragments of the cluster tree representing different types of fractionating curves for Cy5-labelled testis cDNA hybridisation are shown. A. Part of the hierarchical tree with genes having sharp transitions from the hybridised to non-hybridised state near 62% formamide that cluster together. B. Same as A, but with genes that have a sharp transition near 55% formamide. C. Normalised signal intensities (y-axis) over increasing formamide concentrations (x-axis) of the same genes as in A. The vertical line indicates the transition stringency (TS), the midpoint of the transition from hybridized to de-hybridized signal intensities. D. Fractionation curves (x-axis: normalized signal intensities, y-axis: formamide concentration) of the genes shown in B. Vertical line indicates the transition stringency (TS) in this cluster of fractionation curves. E. Cluster of fractionation curves having broad transition regions. F. Fractionation curves of clustering genes having a two-step transition from hybridized to non-hybridized state.

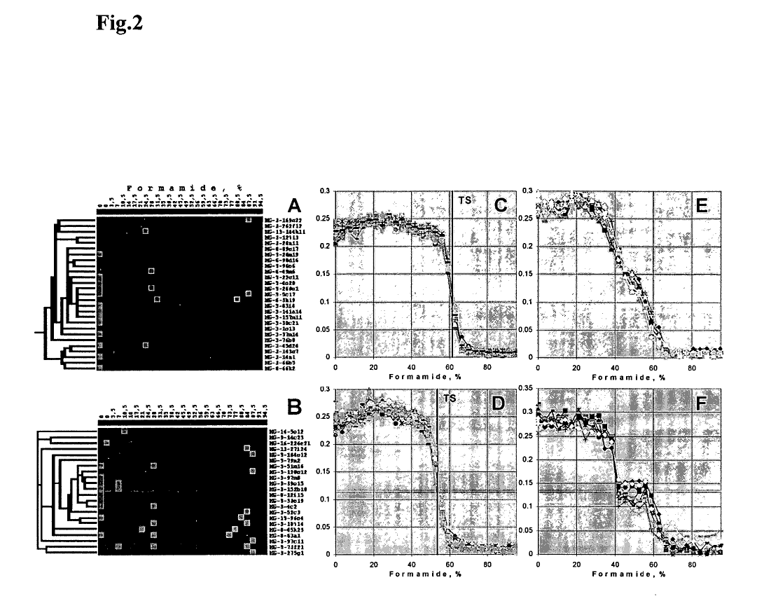

FIG. 3: Transition stringencies are characteristic and reproducible parameters of a probe in combination with specific pools of target molecules. The figure shows the correlation of transition stringencies for two kidney cDNA samples, labelled with Cy3 or Cy5, and hybridised to different slides in independent experiments. The correlation coefficient is 0.95, the standard deviation from the best-fit line for both Cy3 and Cy5 is 1.6% of formamide. Due to the discrete values of transition stringencies in these experiments, random values with uniform distribution from 0 to 1.5 were added to each data point, merely to avoid overlapping data points in the correlation plot. All parameters were calculated from raw data.

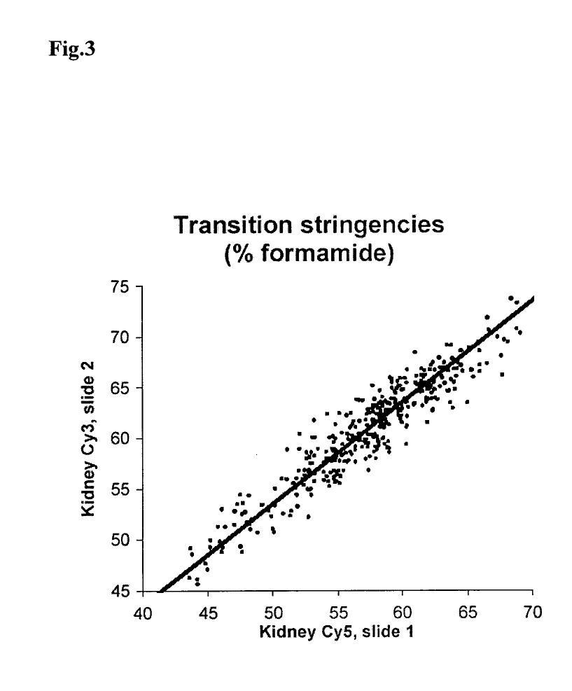

FIG. 4: Using transition stringencies to determine probe specificity. Normalized fractionation curves (A-D) and ratio curves (E, F) for embryo versus adult testis hybridisation in colour flip experiments. A and B show the median of the fractionation curves for all detected spots for embryo versus testis hybridisation. The normalisation was done by subtraction the remaining signal at high stringency such that the median of the last 7 measuring points was put to 0 and multiplying by a scaling factor so that median of first 7 points at high stringency is 1. A. embryo-Cy5 versus testis-Cy3, B. embryo-Cy3 versus testis-Cy5. C to F shows the analysis of transition stringencies for one particular probe, HSP40, in the same experiments. C shows the fractionation curves of HSP40 for the hybridisation experiment shown in A. The green curve (testis-Cy3) shows a shift of the transition region by approximately 20% of formamide to high formamide concentrations as compared to the red curve (embryo-Cy5). The data was normalized by applying the same normalisation factors as in A. D. Normalized HSP40 fractionation curves for the hybridisation experiment shown in B (for embryo-Cy3 versus testis-Cy5). The red curve (testis-Cy5) has a shift of the transition region by approximately 20% of formamide to high concentrations relative to the green curve (embryo-Cy3). Normalized similar to C with the parameters from B. E and F show the ratios of signal intensities measured in C and D, respectively. The curves illustrate the differences in transition stringencies in the two tissues, testis and embryo, for the HSP40 gene.

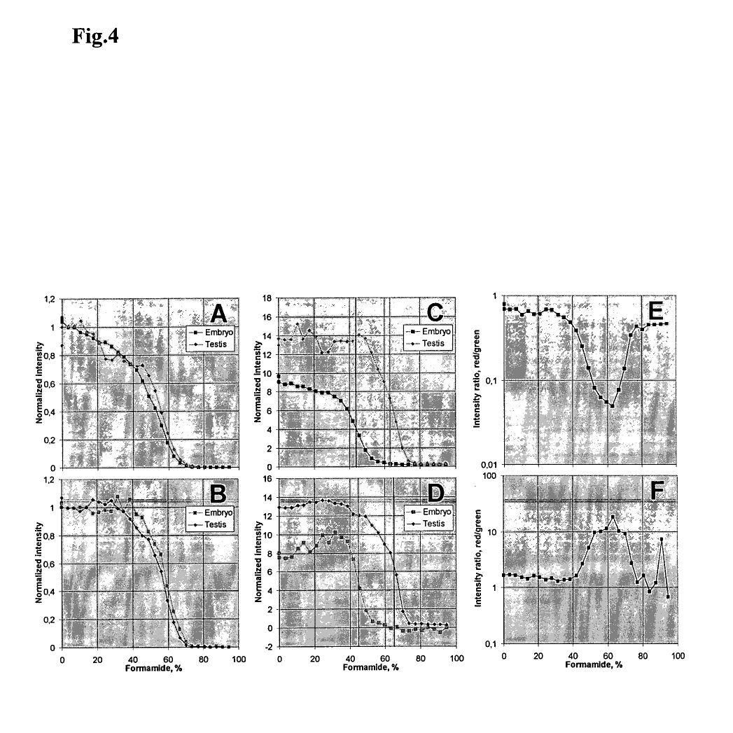

FIG. 5: Quantitative, real-time PCR of HSP40 and HPRT from total RNA of embryo (E10.5, brown lines) and adult testis (blue lines). The house-keeping gene, HPRT, was used as reference (thin, crossed lines). In the exponential amplification phase the background-corrected (subtraction of the value corresponding to the linear signal increase at early cycles) intensity of the HSP40 gene for testis (thick blue line) was 1.9 times higher as compared to the HPRT reference (thin, crossed blue line), while for embryo it was 34 times lower (compare thick brown line and thin, crossed brown line). Thus, the differential expression of HSP40 after normalisation to HPRT is 65 times higher in testis total RNA as compared to embryo total RNA.

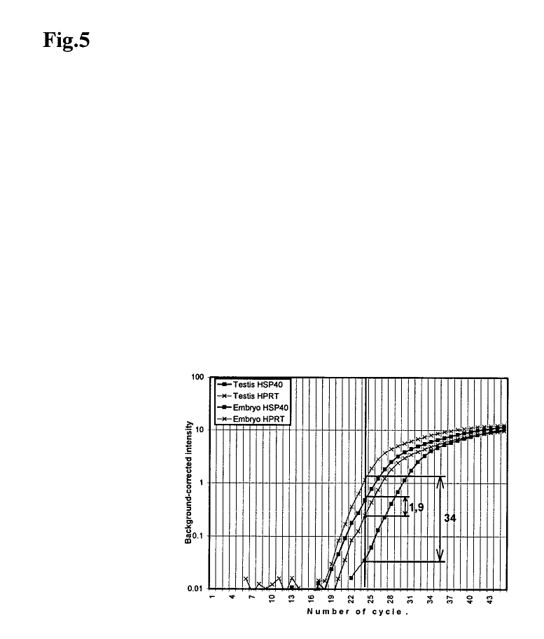

FIG. 6: Summary of genes with decreased transition stringency found in different experiments. Each experiment (#1-#4) consists of two hybridisations (including a colour flip hybridisation) each with simultaneous hybridisation of two different tissues. The genes with decreased transition stringency (referred to as false positives) in both hybridisations are summarised in the first column for each tissue. Some genes were found to be false positives only in one experiment while in the colour flip hybridisation they produced no considerable hybridisation signal (second column). The number of features detected by the image processing software and having a mean signal across the curve above a threshold in both hybridisations is summarised in the third column for each experiment.

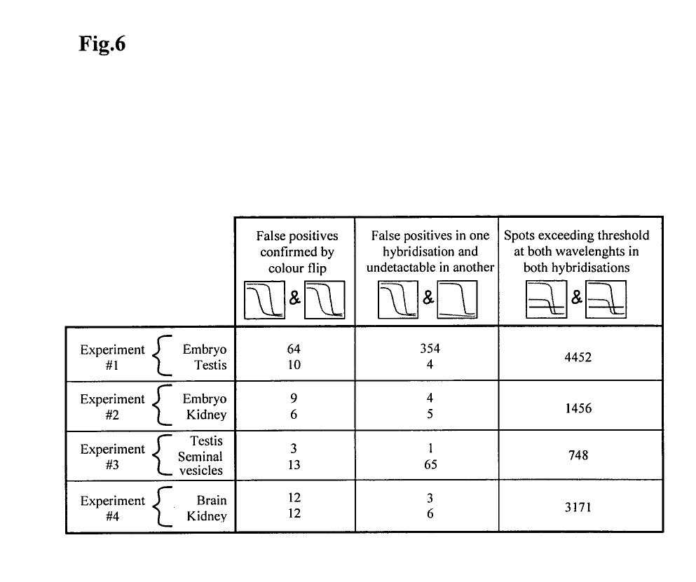

FIG. 7: Correlation plot of the experimentally measured transition stringencies (testis and embryo hybridisation, experiment #1 from FIG. 6) versus the calculated melting temperatures for 22 fully sequenced probes. For nine of them the transition stringencies (TS) were different for embryo and testis RNA samples (white squares, lower TS). Other probes with the same transition stringency are indicated by black squares. The line represents the border between the areas of white and black squares, that is, the border between non-specific and presumably specific areas.

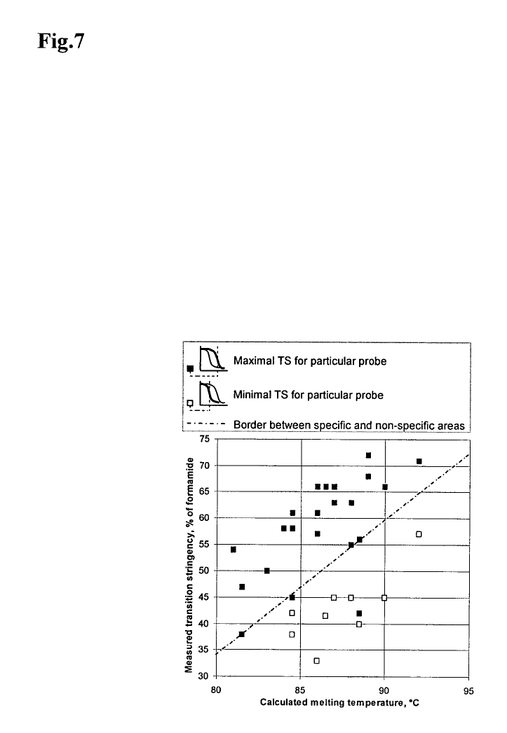

All patents and publications cited above are hereby incorporated herein by reference in their entirety.

1. Method for determining hybridization on a microarray, comprising:
(a) providing a microarray with a plurality of probes;
(b) conducting in situ fractionation of hybridized target in at least one probe of the microarray by means of at least one wash with a defined stringency;
(c) collecting labelling intensity data at or after the in situ fractionation with a defined stringency;
(d) repeating steps (a) and (b), wherein in a subsequent cycle the defined stringency is increased;
(e) generating a set of data corresponding to at least the stringency and the respective labelling intensity data obtained by each cycle for said cycles according to step (c); and
(f) analyzing the set of data for determining hybridization in at least one probe.

2. Method according to claim 1, wherein the labelling intensity data is fluorescent intensity data.

3. Method according to claim 1, wherein step (a) comprises providing a DNA chip.

4. Method according to claim 1 or 3, wherein step (e) comprises generating a fractionation curve.

5. Method according to claim 4, wherein based on characteristic features of the fractionation curve, unreliable data is filtered and eliminated from subsequent analyses.

6. Method according to claim 5, wherein the characteristic features comprise transition stringency.

7. Method according to claim 5, wherein the characteristic features comprise correlation between transition stringency and a calculated temperature of the probe to detect cross-hybridisation.

8. Method according any of the preceding claims, wherein steps (a) to (f) are conducted for a plurality of probes or all probes of said microarray in order to identify probes that produce specific hybridization signals.

9. Method according to any of the preceding claims, with further steps or modified steps as derivable from the remaining specification.

10. Computer program product comprising program code means stored on a computer readable medium for performing the computable part of the method of any of the preceding claims, wherein said program product is capable of being executed by a computer.

11. Computer program product comprising program code means stored on a computer readable medium for performing the computable part of the method of any of the preceding claims, wherein said program product is run on a computer.

12. System for determining hybridization on a microarray, particularly for performing the method of any of claims 1-9, comprising:
(a) a microarray with a plurality of probes;
(b) means for repeatedly conducting in situ fractionation of hybridized target in at least one probe of the microarray by means of at least one wash with a defined stringency;
(c) means for repeatedly collecting fluorescent intensity data at or after the in situ fractionation with a defined stringency;
(d) means for generating a set of data corresponding to at least the stringency and the respective fluorescent intensity data obtained by each cycle for said cycles according to step (c); and
(e) means for analyzing the set of data for determining hybridization in at least one probe.

13. System according to claim 12, wherein the microarray is a DNA chip.

14. System according to claim 12 or 13, wherein a computer is provided to generate a fractionation curve.

15. System according to claim 14, wherein filter means and/or analyzing means are provided for analyzing said fractionation curve in order to filter out unreliable data.

16. System according to any of claims 11-14, with further means or modified means as derivable from the remaining specification.

17. Use of a method according to any of claims 1-9, a computer program product according to claim 10 or 11, and/or a system according to any of claims 12-16 for identifying probes on DNA-chips that produce specific hybridization signals in DNA-chip expression profiling approaches.

18. A method of producing a pharmaceutical composition comprising formulating the compound identified, refined or modified by the method of any of claims 1-9, a computer program product according to claim 10 or 11, and/or a system according to any of claims 12-16, with a pharmaceutically active carrier or diluent.

19. Compound identified, refined or modified by the method of any of claims 1-9, a computer program product according to claim 10 or 11, and/or a system according to any of claims 12-16, with a pharmaceutically active carrier or diluent.
